(19) United States
(12) Patent Application Publication
(10) Pub. No.: US 2022/0133500 A1
(43) Pub. Date: May 5, 2022

(54) INTERVERTEBRAL CAGE FOR FUSION

(71) Applicants: Adi DAGAN, Zichron Yaakov (IL); Amnon YADIN, Kfar Vitkin (IL); Isador LIEBERMAN, Plano, TX (US); Jacob BARAK, Oranit (IL); Agada Medical, Kfar Vitkin (IL)

(72) Inventors: Adi Dagan, Zichron Yaakov (IL); Amnon Yadin, Kfar Vitkin (IL); Isador Lieberman, Plano, TX (US); Jacob Barak, Oranit (IL)

(73) Assignee: Agada Medical, Kfar Vitkin (IL)

(21) Appl. No.: 17/431,532

(22) PCT Filed: Feb. 4, 2020
(86) PCT No.: PCT/US2020/016516

§ 371 Date: Aug. 17, 2021

Related U.S. Application Data
(60) Provisional application No. 62/800,605, filed on Feb. 4, 2019.

(51) Int. Cl. A61F 2/44; A61F 2/30

(52) U.S. Cl. CPC A61F 2/4455; A61F 2/30749; A61F 2002/3092; A61F 2002/3093; A61F 2002/30784; A61B 17/1671; A61F 2310/00071; A61F 2310/00131; A61F 2310/00029; A61F 2310/00059; A61F 2002/302; A61F 2310/00023

(57) ABSTRACT

An intervertebral fusion mechanism includes a disc cage having a scaffolding structure to support bone growth and a porous cancellous bone feeder anchor, connected to the disc cage, for providing a biological material transference interface between cancellous bone and the disc cage.

### PRIORITY INFORMATION

The present application claims priority, under 35 U.S.C. § 119(e), from U.S. Provisional Patent Application, Ser. No. 62/800,605, filed on Feb. 4, 2018. The entire content of U.S. Provisional Patent Application, Ser. No. 62/800,605, filed on Feb. 4, 2018, is hereby incorporated by reference.

### BACKGROUND

A common procedure for handling pain associated with intervertebral discs that have become degenerated due to various factors such as trauma or aging is the use of intervertebral fusion devices for fusing one or more adjacent vertebral bodies. Generally, to fuse the adjacent vertebral bodies, the intervertebral disc is first partially or fully removed. Then, the end plate of the vertebra, the outer layer of the vertebra that was in touch with the removed disc and is made of cortical bone, is scratched to allow for blood and nutrition to flow into the intervertebral space to enhance bone formation, called fusion, between the two vertebral bodies.

Immediately after the scratching of the end plate, an intervertebral fusion device is then typically inserted between neighboring vertebrae to maintain normal disc spacing, lordotic angle, and restore spinal stability throughout the fusion process which takes a few months, thereby facilitating an intervertebral fusion.

There are a number of known conventional fusion devices and methodologies in the art for accomplishing the intervertebral fusion. These include screw and rod arrangements, solid bone implants, and fusion devices usually made of titanium alloys or polyetheretherketone, which include a cage or other implant mechanism which, typically, is packed with bone and/or bone growth inducing substances. These devices are implanted between adjacent vertebral bodies in order to help fuse the vertebral bodies together, in the correct spacing and angle, alleviating the associated pain.

An example of a conventional intervertebral fusion device is disclosed in U.S. Pat. No. 8,845,731. The entire content of U.S. Pat. No. 8,845,731 is hereby incorporated by reference.

As disclosed in U.S. Pat. No. 8,845,731, an expandable fusion device is placed between adjacent vertebral bodies. The fusion device engages the endplates, made of cortical bone, of the adjacent vertebral bodies. When installed, the expandable fusion device facilitates an intervertebral fusion.

Another example of a conventional intervertebral fusion device is disclosed in U.S. Pat. No. 9,402,737. The entire content of U.S. Pat. No. 9,402,737 is hereby incorporated by reference.

As disclosed in U.S. Pat. No. 9,402,737, an intervertebral fusion cage includes an upper component having an outside surface adapted for gripping an upper vertebral endplate and a lower component having an outside surface adapted for gripping a lower vertebral endplate,

A third example of a conventional intervertebral fusion device is disclosed in U.S. Pat. No. 9,801,734. The entire content of U.S. Pat. No. 9,801,734 is hereby incorporated by reference.

As disclosed in U.S. Pat. No. 9,801,734, an expandable spinal fusion implant includes a housing, upper and lower endplates, a wedge positioned within the housing and between the upper and lower endplates, and a drive mechanism to urge the wedge distally between the upper and lower endplates to increase the separation between the endplates,

A further example of a conventional intervertebral fusion device is disclosed in Published US Patent Application Number 2009/0270992. The entire content of Published US Patent Application Number 2009/0270992 is hereby incorporated by reference.

Published US Patent Application Number 2009/0270992 discloses an intervertebral disc that includes spikes for securing the implant to the vertebra and may include an osteoconductive/osteoinductive surface to secure the intervertebral disc to the vertebra.

An additional example of a conventional intervertebral fusion device is disclosed in US Patent Application Number 2014/0309740. The entire content of Published US Patent Application Number 2014/0309740 is hereby incorporated by reference.

US Patent Application Number 2014/0309740 discloses an intervertebral disc implant that includes holes or channels in the surface of the intervertebral disc implant, which interfaces with the vertebra, to promote bone growth to secure the implant to the vertebra.

Lastly, an example of a conventional intervertebral fusion device is disclosed in Published US Patent Application Number 2015/0088258. The entire content of Published US Patent Application Number 2015/0088258 is hereby incorporated by reference.

The various conventional intervertebral fusion mechanisms described above fail to effectively provide a mechanism (scaffold) that encourages and support bone growth within the damaged intervertebral disc or the intervertebral space created by the removal of the intervertebral disc.

Also, the various conventional intervertebral fusion mechanisms described above still show high rated of fusion failure (10%-20%).

More specifically, the various conventional intervertebral fusion mechanisms described above do not effectively provide a fusion cage that enhances the fusion rate and fusion prevalence by integrating scaffold/bone feeder anchors to the cage, wherein the integrated scaffold/bone feeder anchors are in communication with the cancellous bone of the vertebra which is known to be the source of blood, nutrition and cells needed for bone formation.

Therefore, it is desirable to provide an intervertebral fusion cage that effectively creates a transporting/transference channel, conduit, or path that enhances bone growth between the intervertebral space and the cancellous bone to enhance fusion rate and fusion prevalence.

### BRIEF DESCRIPTION OF THE DRAWINGS

The drawings are only for purposes of illustrating various embodiments and are not to be construed as limiting, wherein:

FIG. 1 shows an example of an intervertebral fusion mechanism implanted in the intervertebral space between two adjacent vertebrae;

FIG. 2 shows a top view of the intervertebral fusion mechanism of FIG. 1;

FIG. 3 shows another example of an intervertebral fusion mechanism implanted in the intervertebral space between two adjacent vertebrae;

FIG. 4 shows a third example of an intervertebral fusion mechanism implanted in the intervertebral space between two adjacent vertebrae;

FIG. 5 shows a top view of the intervertebral fusion mechanism of FIG. 5;

FIG. 6 shows a fourth example of an intervertebral fusion mechanism implanted in the intervertebral space between two adjacent vertebrae;

FIG. 7 shows a fifth example of an intervertebral fusion mechanism implanted in the intervertebral space between two adjacent vertebrae;

FIG. 8 shows a top view of the intervertebral fusion mechanism of FIG. 7;

FIG. 9 shows a sixth example of an intervertebral fusion mechanism for implantation in the intervertebral space between two adjacent vertebrae;

FIG. 10 shows the example of FIG. 9 implanted in the intervertebral space between two adjacent vertebrae;

FIG. 11 shows a seventh example of an intervertebral fusion mechanism for implantation in the intervertebral space between two adjacent vertebrae;

FIG. 12 shows the example of FIG. 10 implanted in the intervertebral space between two adjacent vertebrae;

FIG. 13 illustrates another example of an intervertebral fusion mechanism for implantation in the intervertebral space between two adjacent vertebrae;

FIG. 14 illustrates a different view of the intervertebral fusion mechanism of FIG. 13;

FIG. 15 illustrates a different view of the intervertebral fusion mechanism of FIG. 13;

FIG. 16 illustrates another example of an intervertebral fusion mechanism for implantation in the intervertebral space between two adjacent vertebrae wherein cancellous bone feeder anchors are not extended;

FIG. 17 illustrates the intervertebral fusion mechanism of FIG. 16 wherein cancellous bone feeder anchors are extended;

FIG. 18 illustrates another example of an intervertebral fusion mechanism for implantation in the intervertebral space between two adjacent vertebrae wherein cancellous bone feeder anchors are not extended;

FIG. 19 illustrates the intervertebral fusion mechanism of FIG. 18 wherein cancellous bone feeder anchors are extended;

FIGS. 20 and 21 illustrate two views of another example of an intervertebral fusion mechanism for implantation in the intervertebral space between two adjacent vertebrae;

FIGS. 22-24 illustrate three views of example of an intervertebral fusion mechanism for implantation in the intervertebral space between two adjacent vertebrae; and

FIGS. 25 and 26 illustrate another example of an intervertebral fusion mechanism for implantation in the intervertebral space between two adjacent vertebrae.

### DETAILED DESCRIPTION

For a general understanding, reference is made to the drawings. In the drawings, like references have been used throughout to designate identical or equivalent elements. It is also noted that the drawings may not have been drawn to scale and that certain regions may have been purposely drawn disproportionately so that the features and concepts may be properly illustrated.

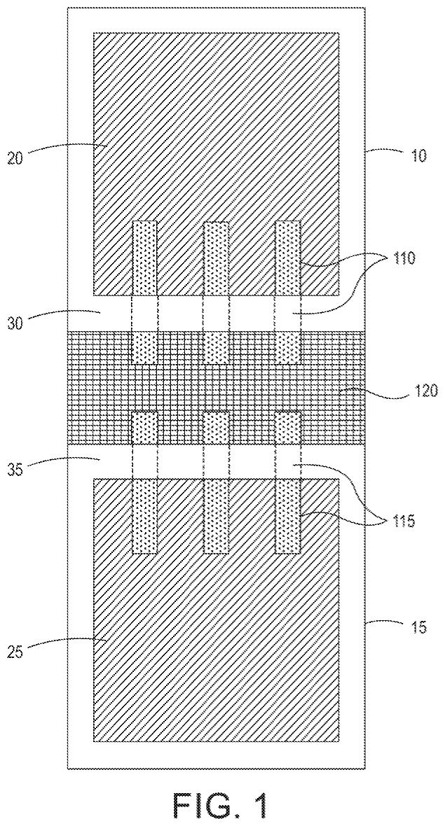

FIG. 1 illustrates an intervertebral fusion mechanism, having a cage 120 and cancellous bone feeder anchors 110 and 115, implanted in an intervertebral space between two adjacent vertebrae 10 and 15. As illustrated in FIG. 1, the cancellous bone feeder anchors 110 extend from the cage 120, through an endplate 30 of first vertebra 10, to the cancellous bone 20 of the first vertebra 10. The cancellous bone feeder anchors 115 extend from the cage 120, through an endplate 35 of second vertebra 15, to the cancellous bone 25 of the second vertebra 15.

It is noted that the cancellous bone feeder anchors, in this embodiment and the other embodiments described below, may be integral with the cage or may be non-integral with the cage.

The cage 120 and the cancellous bone feeder anchors 115 form a fusion device. The cage 120 may include frame members (not shown) to provide strength and support between the two adjacent vertebrae 10 and 15. The cage 120 may also include a void, which can be filled with bone chips, bone patty, etc. to enhance the fusion.

It is noted that the frame members, in this embodiment and the other embodiments described below, may be constructed of porous/scaffold like material to form a porous/scaffold like structure.

In this embodiment and the other embodiments described below, the cage may be constructed of osteoinductive and/or osteoconductive materials to form osteoinductive and/or osteoconductive structure to enhance bone growth. Moreover, in this embodiment and the other embodiments described below, the cage may be covered with and/or include minerals, growth factors, and other biological and chemical components to enhance bone growth.

The cage 120 may include scaffold portions, located between frame members, to provide a transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth in and around the scaffold portions. The scaffold portions of the cage 120 may be formed of titanium or a nickel/titanium alloy or any other material that can serve as a conduit to cells and nutrition.

The cancellous bone feeder anchors 110 and 115 form perforated devices. The cancellous bone feeder anchors 110 and 115 may include structural reinforcement members (not shown) to provide strength. However, the cancellous bone feeder anchors 110 and 115 form an effective porous surface area to interact with the cancellous bone 20, and promote the transfer of blood, nutrition, and cells needed for bone growth. The greater the porous surface area in communication/contact with the cancellous bone, the more effective that the cancellous bone feeder anchors 110 and 115 can promote bone growth in the intervertebral space between the two adjacent vertebrae 10 and 15. The bone growth in and around the feeder anchors and in the intervertebral space between the two adjacent vertebrae 10 and 15 provides strength and support between the disc cage 120 and the two adjacent vertebrae 10 and 15.

As noted above, the cancellous bone feeder anchors 110 and 115 include a perforated structure to provide a scaffold or channel for promoting the transference of nutrients, bone cells, vascular cells, and other biological components from the cancellous bone 20 and 25 to the cage 120 and the volume surrounding the cage 120 so that bone grows in and around the bone feeder anchors 110 and 115 and the cage 120. It is noted that as the bone grows gradually, initially from the bone feeders 110 and 115 towards the cage 120, an early stabilization of the cage 120 is achieved once the bone feeders are consolidated with bone.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone 20 and 25 to the cage 120. In other words, the perforated structure is porous, with interconnecting pores, to promote the transference of nutrients, cells, and other biological components from the cancellous bone 20 and 25 to the perforated structure and the cage 120.

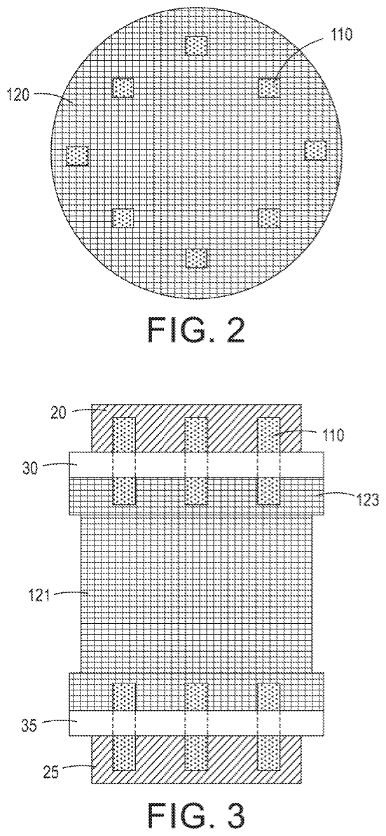

As illustrated in FIG. 2, the cancellous bone feeder anchors 110 are integrated on a surface of the cage 120 so that cancellous bone feeder anchors 110 can extend from (outwardly from the drawing) the cage 120 through the endplate of the vertebra and into the cancellous bone of the vertebra.

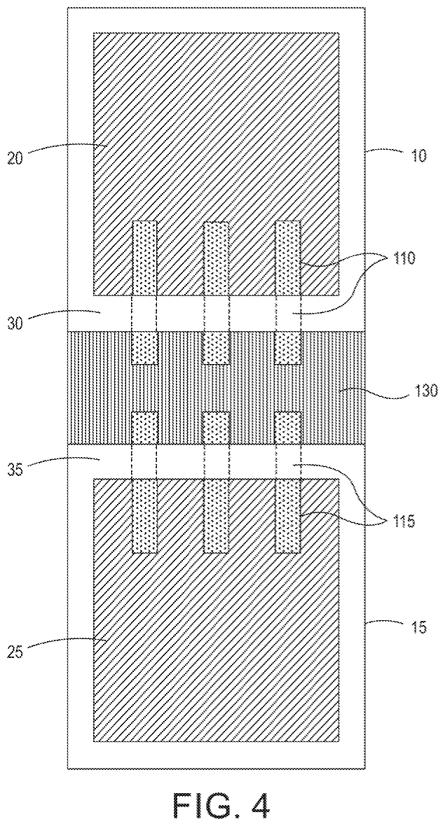

FIG. 3 illustrates another intervertebral fusion mechanism, having a cage 120 and cancellous bone feeder anchors 110, implanted in an intervertebral space between two adjacent vertebrae 10 and 15. As illustrated in FIG. 3, the cancellous bone feeder anchors 110 extend from a cage endplate portion 123, which is an expansion of the cage 120 at the interface with the endplate 30 of first vertebra 10 and an expansion of the cage 120 at the interface with the endplate 35 of second vertebra 15.

It is noted that the cancellous bone feeder anchors 110 may extend from the cage endplate portion 123, originate from within the cage endplate portion 123 and extend through and from the cage endplate portion 123, and/or originate from within the cage 120 and extend through and from the cage endplate portion 123.

The cage endplate portion 123 is a portion of the cage 120 that is reinforced with frame members to provide strength at the interfaces between the cage 120 and the endplates of the vertebrae.

It is noted that that cage endplate portion 123 may be a solid ring surrounding the lattice of the cage 120.

The cage 120 may have scaffold-type portions as discussed above, with respect to FIG. 1. The cage 120 may include solid frame members (not shown) to provide strength and support between the two adjacent vertebrae 10 and 15.

The cage 120 may include scaffold portions, located between frame members, to provide a transportation, connection, and/or support mechanism and for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions.

The scaffolding portions of the cage 120 are formed of titanium or a nickel/titanium alloy or any other material that can serve as a conduit to cells and nutrition.

The cancellous bone feeder anchors 110 and 115 form perforated devices. The cancellous bone feeder anchors 110 and 115 may include structural reinforcement members (not shown) to provide strength. However, the cancellous bone feeder anchors 110 and 115 form an effective porous surface area to interact with the cancellous bone 20, the greater the porous surface area, the more effective that the cancellous bone feeder anchors 110 and 115 can promote bone growth in the intervertebral space between the two adjacent vertebrae 10 and 15. The bone growth in and around the feeder anchors and in the intervertebral space between the two adjacent vertebrae 10 and 15 provides strength and support between the disc cage 120 and the two adjacent vertebrae 10 and 15.

As noted above, the cancellous bone feeder anchors 110 and 115 include a perforated structure to provide a scaffold or channel for promoting the transference of nutrients, bone cells, vascular cells, and other biological components from the cancellous bone 20 and 25 to the cage 120 and the volume surrounding the cage 120 so that bone grows in and around the cage 120.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone 20 and 25 to the cage 120. In other words, the perforated structure is porous, with interconnecting pores, to promote the transference of nutrients, cells, and other biological components from the cancellous bone 20 and 25 to the perforated structure and the cage 120.

FIG. 4 illustrates another intervertebral fusion mechanism, having a cage support frame 130 and cancellous bone feeder anchors 110 and 115, implanted in an intervertebral space between two adjacent vertebrae 10 and 15. As illustrated in FIG. 4, the cancellous bone feeder anchors 110 and 115 extend from a cage (not shown) located within the cage support frame 130.

It is noted that the cancellous bone feeder anchors may extend from the cage and/or originate from within the cage and extend from the cage.

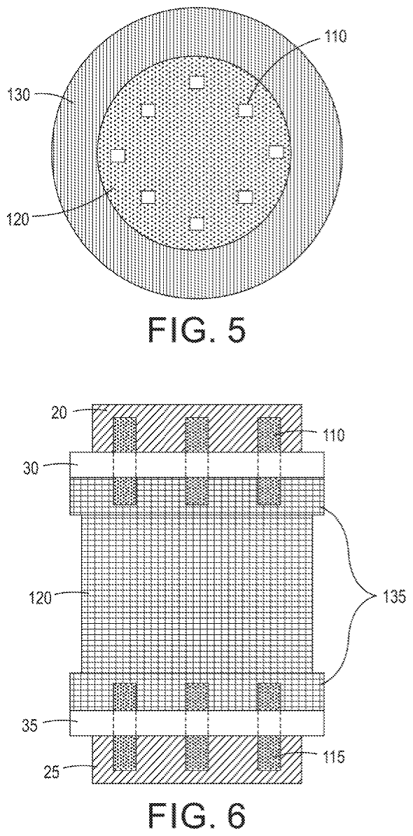

As illustrated in FIG. 5, the cage support frame 130 is a hollow cylinder or ring structure; however, the cage support frame 130 can be many shapes, such as rectangular. The cage support frame 130 provides strength between the endplates 30 and 35 of the vertebrae 10 and 15.

It is noted that the endplates 30 and 35 of the vertebrae 10 and 15 are the ends of the vertebrae that physically touch or contact the intervertebral fusion mechanism after disc removal. The drawings have been purposely drawn disproportionately with respect to this feature.

It is noted that that cage support frame 130 may be a solid structure surrounding the lattice of the cage (not shown).

The cage (not shown) forms a spacer to support intervertebral natural space, lordosis, kyphosis, and general alignment with the whole spine. It includes scaffold-type portions to enhance bone growth and a solid fusion between the adjacent vertebrae. The cage (not shown) may include solid frame members (not shown) to provide strength and support between the two adjacent vertebrae 10 and 15.

The cage (not shown) includes a scaffold (porous Ti or NiTi) portions, and/or voids to be filled with bone chips or other bone growth agents located between frame members, to provide a communication, transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the disc cage 120 are formed of titanium or a nickel/titanium alloy or any other material that can serve as a conduit to cells and nutrition.

The cancellous bone feeder anchors 110 and 115 form perforated devices. The cancellous bone feeder anchors 110 and 115 may include structural reinforcement members (not shown) to provide strength. However, the cancellous bone feeder anchors 110 and 115 form an effective porous surface area to interact with the cancellous bone 20, the greater the porous surface area, the more effective that the cancellous bone feeder anchors 110 and 115 can promote bone growth in the intervertebral space between the two adjacent vertebrae 10 and 15. The bone growth in and around the feeder anchors and in the intervertebral space between the two adjacent vertebrae 10 and 15 provides strength and support between the disc cage 120 and the two adjacent vertebrae 10 and 15.

As noted above, the cancellous bone feeder anchors 110 and 115 include a perforated structure to provide a scaffold or channel for promoting the transference of nutrients, bone cells, vascular cells, and other biological components from the cancellous bone 20 and 25 to the cage 120 and the volume surrounding the cage 120 so that bone grows in and around the cage 120.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone 20 and 25 to the cage (not shown). In other words, the perforated structure is porous, with interconnecting pores, to promote the transference of nutrients, cells, and other biological components from the cancellous bone 20 and 25 to the perforated structure and the cage (not shown).

As illustrated in FIG. 5, the cancellous bone feeder anchors 110 are integrated on a surface of the cage 120, which is surrounded by disc cage support frame 130, so that cancellous bone feeder anchors 110 can extend from (outwardly from the drawing) the cage 120 through the endplate of the vertebra and into the cancellous bone of the vertebra.

It is noted that the cancellous bone feeder anchors may extend from the cage or originate from within the cage and extend from the cage.

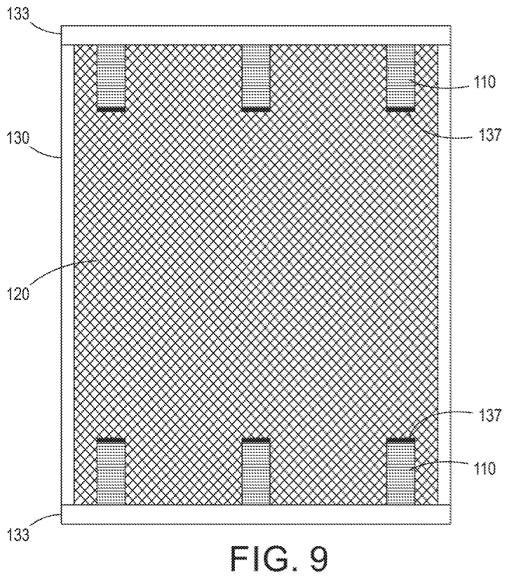

FIG. 6 illustrates another intervertebral fusion mechanism, having a cage support frames 135 and cancellous bone feeder anchors 110 and 115 and integral cage 120, implanted in an intervertebral space between two adjacent vertebrae 10 and 15. As illustrated in FIG. 9, the cancellous bone feeder anchors 110 and 115 extend from the integral cage 120 located between and within the cage support frames 135.

The cage support frames 135 may be hollow cylinders or ring structures that provide strength between the endplates 30 and 35 of the vertebrae 10 and 15.

It is noted that that cage support frames 135 may be solid or perforated structures surrounding the lattice of the cage 120. If the cage support frame is a perforated structure, cancellous bone feeder anchors may be associated with the cage support frame to promote bone growth within the cage support frame.

The cage 120 forms a fusion device. The cage 120 may include solid frame members (not shown) to provide strength and support between the two adjacent vertebrae 10 and 15. The disc cage 120 may also include a void, which can be filled with bone chips, bone patty, etc. to enhance the fusion.

The cage 120 may include scaffold portions, located between frame members, to provide a communication, transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 120 are formed of titanium or a nickel/titanium alloy or any other material that can serve as a conduit to cells and nutrition.

The cancellous bone feeder anchors 110 and 115 form perforated devices. The cancellous bone feeder anchors 110 and 115 may include structural reinforcement members (not shown) to provide strength. However, the cancellous bone feeder anchors 110 and 115 form an effective porous surface area to interact with the cancellous bone 20, the greater the porous surface area, the more effective that the cancellous bone feeder anchors 110 and 115 can promote bone growth in the intervertebral space between the two adjacent vertebrae 10 and 15.

The bone growth in and around the feeder anchors and in the intervertebral space between the two adjacent vertebrae 10 and 15 provides strength and support between the disc cage 120 and the two adjacent vertebrae 10 and 15.

As noted above, the cancellous bone feeder anchors 110 and 115 include a perforated structure to provide a scaffold or channel for promoting the transference of nutrients, bone cells, vascular cells, and other biological components from the cancellous bone 20 and 25 to the cage 120 and the volume surrounding the cage 120 so that bone grows in and around the cage 120.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone 20 and 25 to the cage 120.

In other words, the perforated structure is porous, with interconnecting pores, to promote the transference of nutrients, cells, and other biological components from the cancellous bone 20 and 25 to the perforated structure and the cage 120.

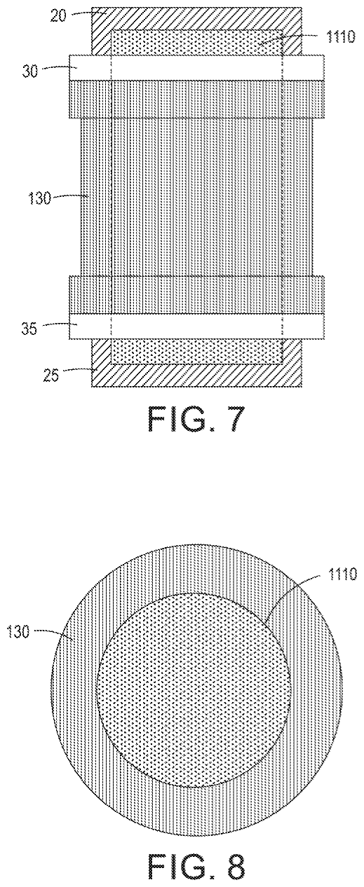

FIG. 7 illustrates another intervertebral fusion mechanism, having a cage support frame 130 and cancellous bone feeder anchor 1110, implanted in an intervertebral space between two adjacent vertebrae 10 and 15. As illustrated in FIG. 7, the cancellous bone feeder anchor 1110 extends through endplates 30 and 35 and into cancellous bone 20 and 25.

It is noted that the cancellous bone feeder anchor may extend from the cage support frame and/or originate from within the cage support frame and extend through and from the cage support frame.

In other words, the cancellous bone feeder anchor 1110 provides both the functionality of providing a support mechanism for growing bone thereon and promoting the transference of nutrients, cells, and other biological components from the cancellous bone 20 and 25. Moreover, the cancellous bone feeder anchor 1110 provides the functionality of anchoring intervertebral fusion mechanism between the two adjacent vertebrae 10 and 15.

In this embodiment, the cancellous bone feeder anchor 1110 located within the cage support frame 130 functions as scaffolding that provides a support mechanism for growing bone thereon as well as frame for supporting the vertebrae until bone growth is realized to complete the fusion process.

On the other hand, in this embodiment, the cancellous bone feeder anchor 1110 located outside the cage support frame 130 promotes the transference of nutrients, cells, and other biological components from the cancellous bone 20 and 25 to the scaffolding located within the cage support frame 130.

The cage support frame 130 is a hollow cylinder or ring structure that provides strength between the endplates 30 and 35 of the vertebrae 10 and 15.

It is noted that the cage support frame 130 is not limited to a hollow cylinder or ring structure but may be any shape, such as a rectangular shape, that provides strength between the endplates 30 and 35 of the vertebrae 10 and 15.

It is further noted that that cage support frame 130 may be a solid structure surrounding the cancellous bone feeder anchor 1110.

The cancellous bone feeder anchor 1110 may include solid frame members (not shown) to provide strength and support between the two adjacent vertebrae 10 and 15.

As illustrated in FIG. 8, the cancellous bone feeder anchor 1110 are integrated with the cage support frame 130 so that cancellous bone feeder anchors 1110 can extend from (outwardly from the drawing) the cage support frame 130 through the endplate of the vertebra and into the cancellous bone of the vertebra.

FIG. 9 illustrates another intervertebral fusion mechanism, having a cage support frame 130, cancellous bone feeder anchors 110 and 115, and integral cage 120, to be implanted in an intervertebral space between two adjacent vertebrae.

The cage support frame 130 is a hollow cylinder or ring structure that provides strength between the endplates of the vertebrae.

It is noted that the cage support frame 130 is not limited to a hollow cylinder or ring structure but may be any shape, such as a rectangular shape, that provides strength between the endplates 30 and 35 of the vertebrae 10 and 15.

It is further noted that that cage support frame 130 may be a solid or perforated structure surrounding the cage 120.

As further illustrated in FIG. 9, the intervertebral fusion mechanism includes a cage support frame endplate interface 133 that interfaces with the endplate of the vertebra. Moreover, the cage support frame endplate interface 133 prevents the cancellous bone feeder anchors 110 from extending from the cage 120 until the state of the cage support frame endplate interface 133 is changed from a closed state to an open state.

The cage support frame endplate interface 133 includes a number of openings, wherein each opening is associated with a cancellous bone feeder anchor 110.

In a closed state, each opening is offset from the associated cancellous bone feeder anchor 110 so that the associated cancellous bone feeder anchor 110 is prevented from extending from the cage 120.

The cage support frame endplate interface 133 may be rotated or shifted, placed in an open state, such that each opening is aligned with the associated cancellous bone feeder anchor 110 so that the associated cancellous bone feeder anchor 110 can extend from the cage 120.

The cancellous bone feeder anchors 110 may be constructed to have spring functionality or a spring-like characteristic so that when the cage support frame endplate interface 133 is rotated or shifted into the open state, the spring functionality or spring-like characteristic of the cancellous bone feeder anchors 110 causes the cancellous bone feeder anchors 110 to expand and extend from the cage 120.

It is noted that another mechanism for extending the cancellous bone feeder anchors 110 to the cancellous bone is a balloon. By inflating a deflated balloon, located within the cage 120, the balloon pushes the cancellous bone feeder anchors 110 into the cancellous bone.

The cage 120, as shown in FIG. 9, may include cancellous bone feeder anchor bases 137 which provide a backstop for the cancellous bone feeder anchors, thereby forcing the cancellous bone feeder anchors 110, during open state expansion, to extend from the cage 120 and not into the cage 120.

The cage 120 forms a fusion device. The cage 120 may include solid frame members (not shown) to provide strength and support between the two adjacent vertebrae. The disc cage 120 may also include a void, which can be filled with bone chips, bone patty, etc. to enhance the fusion.

It is noted that cancellous bone feeder anchor bases 137 may be integral with the frame members of the cage 120.

The cage 120 may include scaffold portions, located between frame members, to provide a communication, transportation, connection, and/or support mechanism for growing bone thereon. The scaffold portions are constructed to create a plurality of voids (holes or pores) where bone can grow therein so that the scaffold, when the bone is completely formed therearound, is located within the formed bone.

In other words, the scaffold portions are porous, and the pores may be interconnected, to promote bone growth around the scaffolding portions. The scaffolding portions of the cage 120 are formed of titanium or a nickel/titanium or any other material that can serve as a conduit to cells and nutrition.

The cancellous bone feeder anchors 110 and 115 form perforated devices. The cancellous bone feeder anchors 110 and 115 may include structural reinforcement members (not shown) to provide strength. However, the cancellous bone feeder anchors 110 and 115 form an effective porous surface area to interact with the cancellous bone 20, the greater the porous surface area, the more effective that the cancellous bone feeder anchors 110 and 115 can promote bone growth in the intervertebral space between the two adjacent vertebrae 10 and 15. The bone growth in and around the feeder anchors and in the intervertebral space between the two adjacent vertebrae 10 and 15 provides strength and support between the disc cage 120 and the two adjacent vertebrae 10 and 15.

As noted above, the cancellous bone feeder anchors 110 and 115 include a perforated structure to provide a scaffold or channel for promoting the transference of nutrients, bone cells, vascular cells, and other biological components from the cancellous bone 20 and 25 to the cage 120 and the volume surrounding the cage 120 so that bone grows in and around the cage 120.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone 20 and 25 to the cage 120. In other words, the perforated structure is porous, with interconnecting pores, to promote the transference of nutrients, cells, and other biological components from the cancellous bone 20 and 25 to the perforated structure and the cage 120.

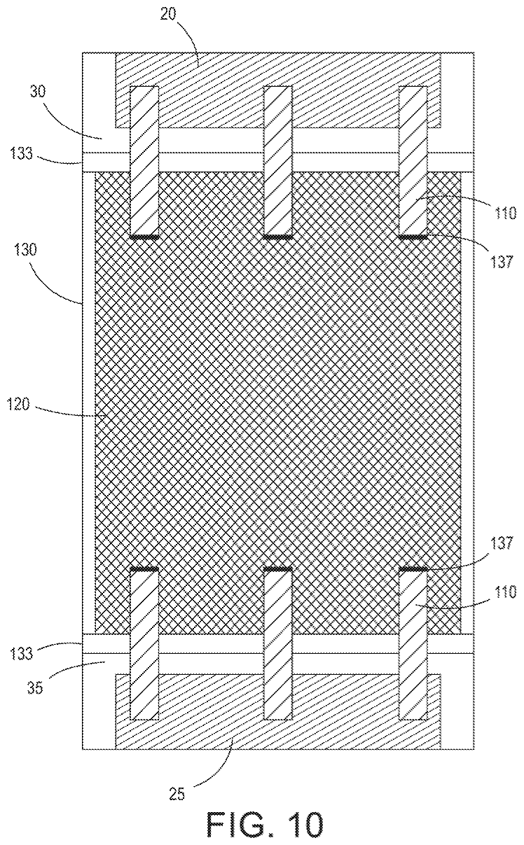

FIG. 10 illustrates the intervertebral fusion mechanism of FIG. 9, wherein the cage support frame endplate interface 133 has been rotated or shifted into an open state and the cancellous bone feeder anchors 110 have expanded and extended from the cage 120, through the endplates 30 and 35, and into the cancellous bone 20 and 25.

It is noted that the mechanism for retaining and releasing (extending) cancellous bone feeder anchors is not limited to the illustrated the cage support frame endplate interface but may be any mechanism or mechanism keeps the cancellous bone feeder anchors in a state or location to enable proper and effective insertion of the cage between the vertebrae and once the cage is properly located between the vertebrae, the cancellous bone feeder anchors can be extended (moved) into the cancellous bone.

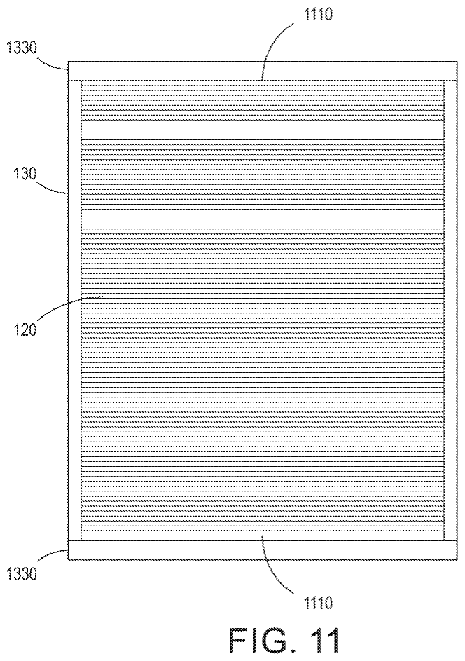

FIG. 11 illustrates another intervertebral fusion mechanism, having a cage support frame 130 and cancellous bone feeder anchor 1110, to be implanted in an intervertebral space between two adjacent vertebrae.

The cage support frame 130 is a hollow cylinder or ring structure that provides strength between the endplates of the vertebrae.

It is noted that the cage support frame 130 is not limited to a hollow cylinder or ring structure but may be any shape, such as a rectangular shape, that provides strength between the endplates 30 and 35 of the vertebrae 10 and 15.

It is noted that that cage support frame 130 may be a solid or perforated structure surrounding the scaffolding of the cage 120.

As further illustrated in FIG. 11, the intervertebral fusion mechanism includes a cage support frame endplate interface 1330 that interfaces with the endplate of the vertebra. Moreover, the cage support frame endplate interface 1330 prevents the cancellous bone feeder anchor 1110 from extending until the cage support frame endplate interface 133 is removed.

The cage support frame endplate interface 133 is removed so that the cancellous bone feeder anchor 1110 can extend through an endplate and into a cancellous bone.

It is noted that the cancellous bone feeder anchor 1110 may be constructed of a memory shape alloy, such as NiTi to enable the extension of the cancellous bone feeder anchor 1110 into the cancellous bone.

The cancellous bone feeder anchor 1110 may be constructed to have spring functionality or a spring-like characteristic so that when the cage support frame endplate interface 133 is removed, the spring functionality or spring-like characteristic of the cancellous bone feeder anchor 1110 causes the cancellous bone feeder anchor 1110 to expand and extend through an endplate and into a cancellous bone.

It is noted that the endplate/cancellous bone of the vertebra may be predrilled so that the cancellous bone feeder anchor 1110 will expand (extend) into the predrilled hole.

Moreover, the cancellous bone feeder anchor 1110 provides the functionality of anchoring intervertebral fusion mechanism between the two adjacent vertebrae.

In this embodiment, the cancellous bone feeder anchor 1110 located within the cage support frame 130 functions as scaffolding that provides a support mechanism for growing bone thereon as well as frame for supporting the vertebrae until bone growth is realized to complete the fusion process.

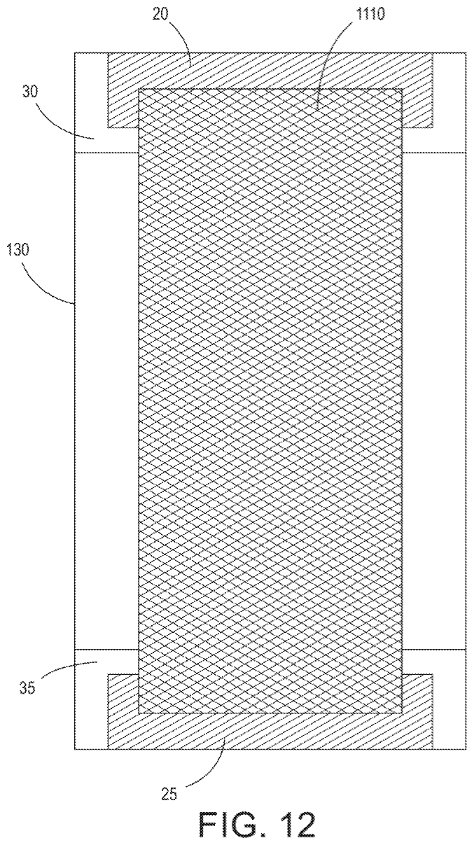

FIG. 12 illustrates the intervertebral fusion mechanism of FIG. 11, wherein the cage support frame endplate interface 133 has been removed, and the cancellous bone feeder anchor 1110 has expanded and extended through the endplates 30 and 35 and into the cancellous bone 20 and 25.

In this embodiment, the cancellous bone feeder anchor 1110 located within the cage support frame 130 functions as scaffolding that provides a support mechanism for growing bone thereon as well as frame for supporting the vertebrae until bone growth is realized to complete the fusion process.

On the other hand, in this embodiment, the cancellous bone feeder anchor 1110 located outside the cage support frame 130 promotes the transference of nutrients, cells, and other biological components from the cancellous bone 20 and 25 to the scaffolding located within the cage support frame 130.

As illustrated in FIG. 12, the integral intervertebral fusion mechanism includes a scaffold portion for enabling the growth of new bone in the volume that once housed the disc (intervertebral space). The scaffold portion is a continuous structure from the cancellous bone of one vertebra to the cancellous bone in the adjacent vertebra. The continuous scaffold structure provides a mechanism for enabling the transportation of nutrients, cells, and other biological components from the cancellous bone and the promotion of bone growth between the adjacent vertebrae.

In the various embodiments discussed above, the integral intervertebral fusion mechanisms are constructed using 3-D printing. 3-D printing enables the integral construction of the various intervertebral fusion mechanisms described above.

Moreover, 3-D printing enables the construction of different portions with different porosity, pore sizes, designs, and shapes. Additionally, 3-D printing enables construction on a micro and macro scale.

Moreover, the micro structure of the cancellous bone and/or the cortical bone of the vertebra of the patient can be mapped from the patient's imaging studies and duplicated in the 3-D printing process so that the anchors as well as portions of the cage can match the same structure and further enhance bone growth.

In preparing the intervertebral space, channels (holes) are drilled into the endplate of the vertebra at locations corresponding to the cancellous bone feeder anchors of the integral intervertebral fusion mechanism.

If the integral intervertebral fusion mechanism of FIGS. 7, 8, 11, and 12 are utilized, a single channel (hole) is drilled into the endplate of the vertebra.

More specifically, the topography of the endplates of two adjacent vertebrae is mapped. An intervertebral fusion mechanism is constructed, using 3-D printing, so that the geometry and topography of intervertebral fusion mechanism endplate, on the macro and micro scales, match the desired intervertebral space geometry and the topography of the associated mapped endplates and cancellous bone of the vertebrae.

Thereafter, the intervertebral fusion mechanism is inserted between the adjacent vertebrae. Once the integral intervertebral fusion mechanism is properly located between the adjacent vertebrae, the cancellous bone feeder anchor mechanism(s) are released so that the cancellous bone feeder anchor mechanism(s) can expand and extend through the endplate of the vertebrae and into the cancellous bone.

In summary, an intervertebral fusion mechanism is provided to provide both the functionality of providing a support mechanism for growing bone thereon and promoting the transference of nutrients, cells, and other biological components from the cancellous bone. Moreover, the intervertebral fusion mechanism provides an anchoring functionality to secure the fusion device between the vertebrae.

It is noted that the intervertebral fusion mechanism may be integrally constructed.

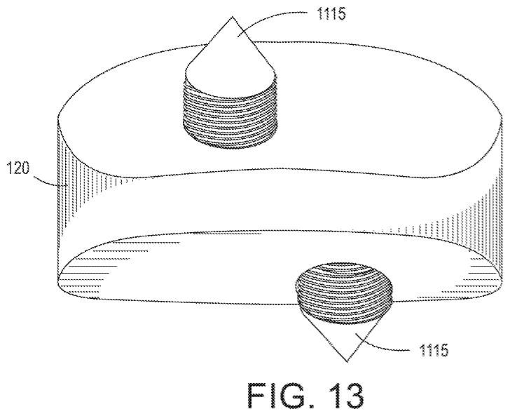

FIG. 13 illustrates another example of an intervertebral fusion mechanism for implantation in the intervertebral space between two adjacent vertebrae. As illustrated in FIG. 13, the intervertebral fusion mechanism includes a cage 120 and cancellous bone feeder anchors 1115. In this embodiment, the cancellous bone feeder anchors 1115 are configured to provide a drilling function such that as the cancellous bone feeder anchors 1115 are rotated, the cancellous bone feeder anchors 1115 travel (drill) into the endplate of the vertebra as well as the cancellous bone of the vertebra.

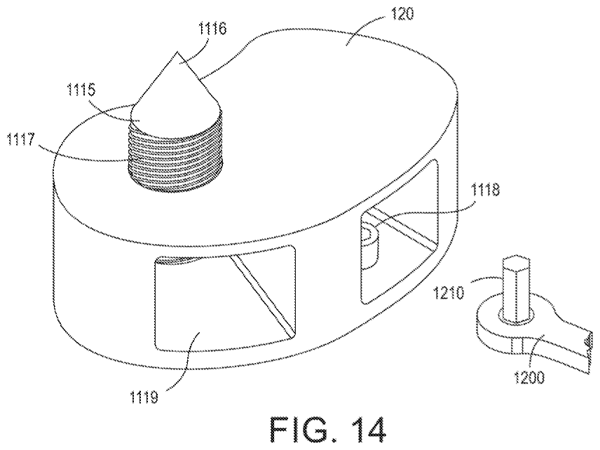

FIG. 14 illustrates a different view of the intervertebral fusion mechanism of FIG. 13. As illustrated in FIG. 14, the intervertebral fusion mechanism includes a cage 120 and cancellous bone feeder anchors 1115. In this embodiment, as illustrated, the cancellous bone feeder anchors 1115 are configured to include a drill bit head 1116 to provide a drilling function. The cancellous bone feeder anchors 1115 may include threads 1117 to facilitate the drilling function.

As further illustrated, the cancellous bone feeder anchors 1115 include a drive receptacle 1118 to receive a drive mechanism 1210 of a drive tool 1200, and the cage includes an opening 1119 to allow the drive mechanism 1210 of the drive tool 1200 access to the drive receptacle 1118 of the cancellous bone feeder anchors 1115.

As the drive mechanism 1210 of the drive tool 1200 is rotated in a first direction, the cancellous bone feeder anchors 1115 are rotated so that the cancellous bone feeder anchors 1115 travel (drill) into the endplate of the vertebra as well as the cancellous bone of the vertebra. It is noted that if the drive mechanism 1210 of the drive tool 1200 is rotated in a second direction, opposite the first direction, the cancellous bone feeder anchors 1115 will travel out of (retreat from) the endplate of the vertebra as well as the cancellous bone of the vertebra.

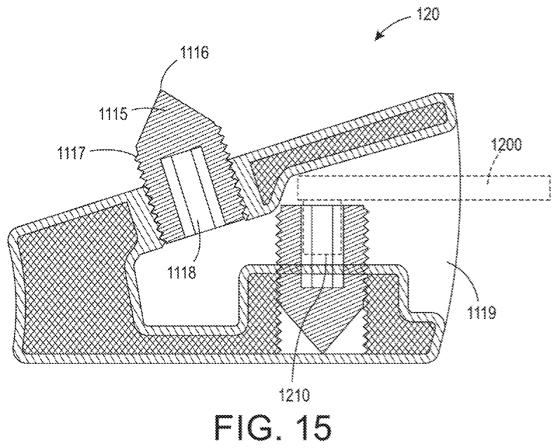

FIG. 15 illustrates a different view of the intervertebral fusion mechanism of FIG. 13. As illustrated in FIG. 15, the intervertebral fusion mechanism includes a cage 120 and cancellous bone feeder anchors 1115. In this embodiment, as illustrated, the cancellous bone feeder anchors 1115 are configured to include a drill bit head 1116 to provide a drilling function. The cancellous bone feeder anchors 1115 may include threads 1117 to facilitate the drilling function.

As further illustrated, the cancellous bone feeder anchors 1115 include a drive receptacle 1118 to receive a drive mechanism 1210 of a drive tool 1200, and the cage includes an opening 1119 to allow the drive mechanism 1210 of the drive tool 1200 access to the drive receptacle 1118 of the cancellous bone feeder anchors 1115.

As the drive mechanism 1210 of the drive tool 1200 is rotated in a first direction, the cancellous bone feeder anchors 1115 are rotated so that the cancellous bone feeder anchors 1115 travel (drill) into the endplate of the vertebra as well as the cancellous bone of the vertebra. It is noted that if the drive mechanism 1210 of the drive tool 1200 is rotated in a second direction, opposite the first direction, the cancellous bone feeder anchors 1115 will travel out of (retreat from) the endplate of the vertebra as well as the cancellous bone of the vertebra.

It is noted that the illustrated drive mechanism 1210 and the drive tool 1200 show an orthogonal configuration; however, the drive receptacle 1118 could be constructed with a flexible cable such that the drive mechanism 1210 and the drive tool 1200 have a co-linear (straight line) configuration. In this embodiment, as the drive mechanism 1210 of the drive tool 1200 rotates the flexible cable, through the drive receptacle 1118, the flexible cable rotates the cancellous bone feeder anchor 1115.

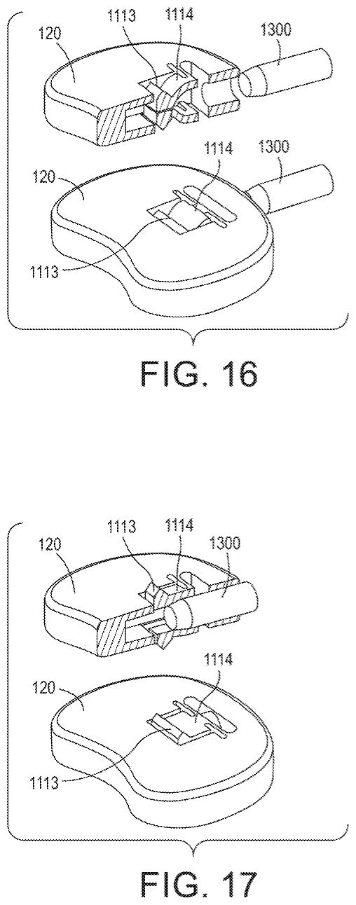

FIG. 16 illustrates another example of an intervertebral fusion mechanism for implantation in the intervertebral space between two adjacent vertebrae wherein cancellous bone feeder anchors are not extended. As illustrated in FIG. 16, the intervertebral fusion mechanism includes a cage 120 and cancellous bone feeder anchors 1113, wherein the cancellous bone feeder anchors 1113 are in a non-extended state.

The cage 120 includes cancellous bone feeder anchor extension mechanisms 1114, which translate a force from an extension tool 1300 to a force that forces the cancellous bone feeder anchors 1113 to extend from the cage 120.

In FIG. 16, the cancellous bone feeder anchor extension mechanisms 1114 are arc shaped members that rotate such that as a force is applied to the members, the members move in a direction to cause the cancellous bone feeder anchors 1113 to extend from the cage 120.

It is noted that cancellous bone feeder anchor extension mechanism 1114 and cancellous bone feeder anchor 1113 may be constructed as an integral piece.

FIG. 17 illustrates the intervertebral fusion mechanism of FIG. 16 wherein cancellous bone feeder anchors are extended. As illustrated in FIG. 17, the intervertebral fusion mechanism includes a cage 120 and cancellous bone feeder anchors 1113, wherein the cancellous bone feeder anchors 1113 are in a non-extended state.

The cage 120 includes cancellous bone feeder anchor extension mechanisms 1114, which translate a force from an extension tool 1300 to a force that forces the cancellous bone feeder anchors 1113 to extend from the cage 120.

In FIG. 17, the cancellous bone feeder anchor extension mechanisms 1114 are arc shaped members that have been rotated such that the cancellous bone feeder anchors 1113 have been extended from the cage 120.

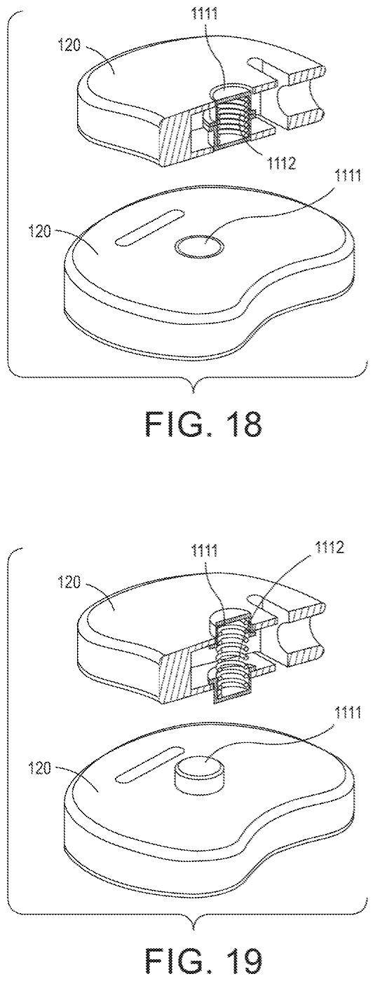

FIG. 18 illustrates another example of an intervertebral fusion mechanism for implantation in the intervertebral space between two adjacent vertebrae wherein cancellous bone feeder anchors are not extended. As illustrated in FIG. 18, the intervertebral fusion mechanism includes a cage 120 and cancellous bone feeder anchors 1111, wherein the cancellous bone feeder anchors 1111 are in a non-extended state.

The cage 120 includes cancellous bone feeder anchor extension mechanisms (springs) 1112, which, when released, force the cancellous bone feeder anchors 1111 to extend from the cage 120.

FIG. 19 illustrates the intervertebral fusion mechanism of FIG. 18 wherein cancellous bone feeder anchors are extended. As illustrated in FIG. 19, the intervertebral fusion mechanism includes a cage 120 and cancellous bone feeder anchors 1111, wherein the cancellous bone feeder anchors 1111 are in a non-extended state.

The cage 120 includes cancellous bone feeder anchor extension mechanisms (springs) 1112, which have been released, forcing the cancellous bone feeder anchors 1111 to extend from the cage 120.

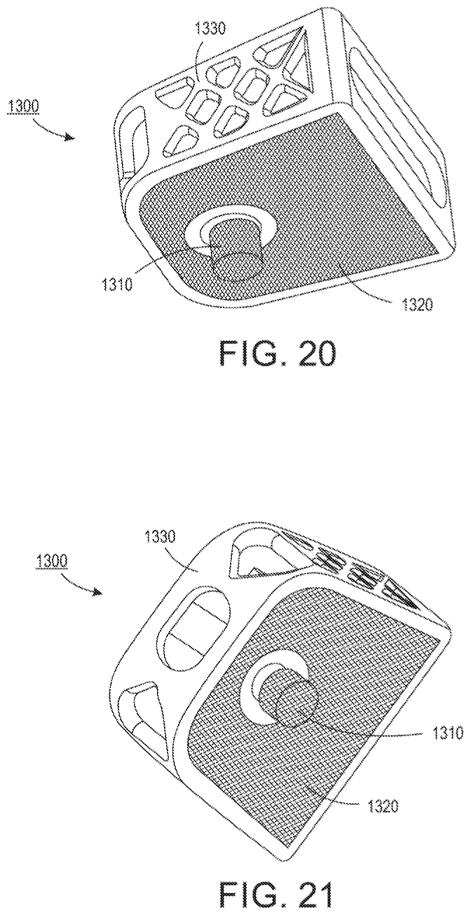

FIG. 20 illustrates one view of another example of an intervertebral fusion mechanism 1300 for implantation in the intervertebral space between two adjacent vertebrae. As illustrated in FIG. 20, the intervertebral fusion mechanism 1300 includes a cage 1330, cancellous bone feeder anchor 1310, and a scaffold structure 1320 on a surface of the cage 1330 that engages the endplate of the vertebra. The cage 1330 may have openings or voids, as illustrated, which can be filled with bone chips, bone patty, etc. to enhance the fusion.

The cage 1330 forms a fusion device. The cage 1330 may include solid frame members (not shown) to provide strength and support between the two adjacent vertebrae.

It is noted that cancellous bone feeder anchor bases 1310 may be integral with the frame members of the cage 1330.

The cage 1330 may include scaffold portions, located between frame members, to provide a transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 120 are formed of titanium or a nickel/titanium or any other material that can serve as a conduit to cells and nutrition.

The cancellous bone feeder anchor 1310 forms perforated devices. The cancellous bone feeder anchor 1310 may include structural reinforcement members (not shown) to provide strength. However, the cancellous bone feeder anchor 1310 forms an effective porous surface area to interact with the cancellous bone, the greater the porous surface area, the more effective that the cancellous bone feeder anchor 1310 can promote bone growth in the intervertebral space between the two adjacent vertebrae. The bone growth in and around the feeder anchor and in the intervertebral space between the two adjacent vertebrae provides strength and support between the disc cage 1330 and the two adjacent vertebrae.

As noted above, the cancellous bone feeder anchor 1310 includes a perforated structure to provide a scaffold or channel for promoting the transference of nutrients, bone cells, vascular cells, and other biological components from the cancellous bone to the cage 1330 and the volume surrounding the cage 1330 so that bone grows in and around the cage 1330.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1330. In other words, the perforated structure is porous, with interconnecting pores, to promote the transference of nutrients, cells, and other biological components from the cancellous bone to the perforated structure and the cage 1330.

FIG. 21 illustrates another view of the intervertebral fusion mechanism 1300 of FIG. 20. As illustrated in FIG. 21, the intervertebral fusion mechanism 1300 includes a cage 1330, cancellous bone feeder anchor 1310, and a scaffold structure 1320 on a surface of the cage 1330 that engages the endplate of the vertebra. The cage 1330 may have openings or voids, as illustrated, which can be filled with bone chips, bone patty, etc. to enhance the fusion.

The cage 1330 forms a fusion device. The cage 1330 may include solid frame members (not shown) to provide strength and support between the two adjacent vertebrae.

It is noted that cancellous bone feeder anchor bases 1310 may be integral with the frame members of the cage 1330.

The cage 1330 may include scaffold portions, located between frame members, to provide a communication, transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 1330 are formed of titanium or a nickel/titanium or any other material that can serve as a conduit to cells and nutrition.

The cancellous bone feeder anchor 1310 forms perforated devices. The cancellous bone feeder anchor 1310 may include structural reinforcement members (not shown) to provide strength. However, the cancellous bone feeder anchor 1310 forms an effective porous surface area to interact with the cancellous bone, the greater the porous surface area, the more effective that the cancellous bone feeder anchor 1310 can promote bone growth in the intervertebral space between the two adjacent vertebrae. The bone growth in and around the feeder anchor and in the intervertebral space between the two adjacent vertebrae provides strength and support between the disc cage 1330 and the two adjacent vertebrae.

As noted above, the cancellous bone feeder anchor 1310 includes a perforated structure to provide a scaffold or channel for promoting the transference of nutrients, bone cells, vascular cells, and other biological components from the cancellous bone to the cage 1330 and the volume surrounding the cage 1330 so that bone grows in and around the cage 1330.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1330. In other words, the perforated structure is porous, with interconnecting pores, to promote the transference of nutrients, cells, and other biological components from the cancellous bone to the perforated structure and the cage 1330.

FIG. 22 illustrates one view of another example of an intervertebral fusion mechanism 1400 for implantation in the intervertebral space between two adjacent vertebrae. As illustrated in FIG. 22, the intervertebral fusion mechanism 1400 includes a cage 1430, cancellous bone feeder anchors 1410, a scaffold structure 1420 on a surface of the cage 1430 that engages the endplate of the vertebra, and a spring mechanism 1440 to bias the cancellous bone feeder anchors 1410 into the cancellous bone.

The cage 1430 may have openings or voids, as illustrated, which can be filled with bone chips, bone patty, etc. to enhance the fusion.

The cage 1430 forms a fusion device. The cage 1430 may include solid frame members (not shown) to provide strength and support between the two adjacent vertebrae.

It is noted that cancellous bone feeder anchor bases 1410 may be integral with the frame members of the cage 1430.

The cage 1430 may include scaffold portions, located between frame members, to provide a transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 1430 are formed of titanium or a nickel/titanium or any other material that can serve as a conduit to cells and nutrition.

The cancellous bone feeder anchors 1410 form perforated devices. The cancellous bone feeder anchors 1410 may include structural reinforcement members (not shown) to provide strength. However, the cancellous bone feeder anchors 1410 form an effective porous surface area to interact with the cancellous bone, the greater the porous surface area, the more effective that the cancellous bone feeder anchors 1410 can promote bone growth in the intervertebral space between the two adjacent vertebrae. The bone growth in and around the feeder anchor and in the intervertebral space between the two adjacent vertebrae provides strength and support between the disc cage 1430 and the two adjacent vertebrae.

As noted above, the cancellous bone feeder anchors 1410 include a perforated structure to provide a scaffold or channel for promoting the transference of nutrients, bone cells, vascular cells, and other biological components from the cancellous bone to the cage 1430 and the volume surrounding the cage 1430 so that bone grows in and around the cage 1430.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1430. In other words, the perforated structure is porous, with interconnecting pores, to promote the transference of nutrients, cells, and other biological components from the cancellous bone to the perforated structure and the cage 1430.

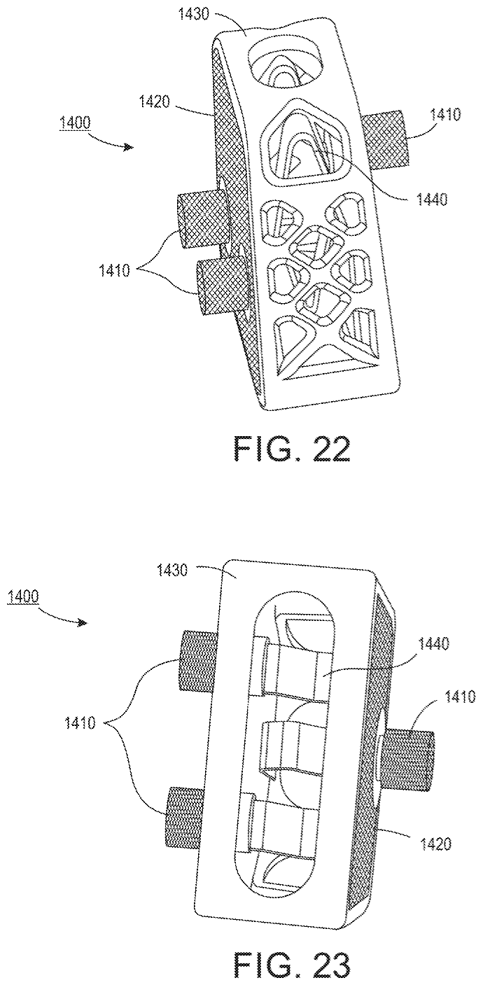

FIG. 23 illustrates one view of another example of an intervertebral fusion mechanism 1400 for implantation in the intervertebral space between two adjacent vertebrae. As illustrated in FIG. 23, the intervertebral fusion mechanism 1400 includes a cage 1430, cancellous bone feeder anchors 1410, a scaffold structure 1420 on a surface of the cage 1430 that engages the endplate of the vertebra, and a spring mechanism 1440 to bias the cancellous bone feeder anchors 1410 into the cancellous bone.

The cage 1430 may have openings or voids, as illustrated, which can be filled with bone chips, bone patty, etc. to enhance the fusion.

The cage 1430 forms a fusion device. The cage 1430 may include solid frame members (not shown) to provide strength and support between the two adjacent vertebrae.

It is noted that cancellous bone feeder anchor bases 1410 may be integral with the frame members of the cage 1430.

The cage 1430 may include scaffold portions, located between frame members, to provide a communication, transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 1430 are formed of titanium or a nickel/titanium or any other material that can serve as a conduit to cells and nutrition.

The cancellous bone feeder anchors 1410 form perforated devices. The cancellous bone feeder anchors 1410 may include structural reinforcement members (not shown) to provide strength. However, the cancellous bone feeder anchors 1410 form an effective porous surface area to interact with the cancellous bone, the greater the porous surface area, the more effective that the cancellous bone feeder anchors 1410 can promote bone growth in the intervertebral space between the two adjacent vertebrae.

The bone growth in and around the feeder anchor and in the intervertebral space between the two adjacent vertebrae provides strength and support between the disc cage 1430 and the two adjacent vertebrae.

As noted above, the cancellous bone feeder anchors 1410 include a perforated structure to provide a scaffold or channel for promoting the transference of nutrients, bone cells, vascular cells, and other biological components from the cancellous bone to the cage 1430 and the volume surrounding the cage 1430 so that bone grows in and around the cage 1430.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1430. In other words, the perforated structure is porous, with interconnecting pores, to promote the transference of nutrients, cells, and other biological components from the cancellous bone to the perforated structure and the cage 1430.

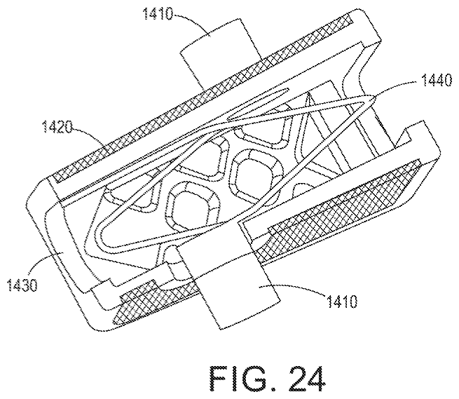

FIG. 24 illustrates one view of another example of an intervertebral fusion mechanism 1400 for implantation in the intervertebral space between two adjacent vertebrae. As illustrated in FIG. 24, the intervertebral fusion mechanism 1400 includes a cage 1430, cancellous bone feeder anchors 1410, a scaffold structure 1420 on a surface of the cage 1430 that engages the endplate of the vertebra, and a spring mechanism 1440 to bias the cancellous bone feeder anchors 1410 into the cancellous bone.

The cage 1430 may have openings or voids, as illustrated, which can be filled with bone chips, bone patty, etc. to enhance the fusion.

The cage 1430 forms a fusion device. The cage 1430 may include solid frame members (not shown) to provide strength and support between the two adjacent vertebrae.

It is noted that cancellous bone feeder anchor bases 1410 may be integral with the frame members of the cage 1430.

The cage 1430 may include scaffold portions, located between frame members, to provide a transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth around the scaffolding portions. The scaffolding portions of the cage 1430 are formed of titanium or a nickel/titanium or any other material that can serve as a conduit to cells and nutrition.

The cancellous bone feeder anchors 1410 form perforated devices. The cancellous bone feeder anchors 1410 may include structural reinforcement members (not shown) to provide strength. However, the cancellous bone feeder anchors 1410 form an effective porous surface area to interact with the cancellous bone, the greater the porous surface area, the more effective that the cancellous bone feeder anchors 1410 can promote bone growth in the intervertebral space between the two adjacent vertebrae. The bone growth in and around the feeder anchor and in the intervertebral space between the two adjacent vertebrae provides strength and support between the disc cage 1430 and the two adjacent vertebrae.

As noted above, the cancellous bone feeder anchors 1410 include a perforated structure to provide a scaffold or channel for promoting the transference of nutrients, bone cells, vascular cells, and other biological components from the cancellous bone to the cage 1430 and the volume surrounding the cage 1430 so that bone grows in and around the cage 1430.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1430. In other words, the perforated structure is porous, with interconnecting pores, to promote the transference of nutrients, cells, and other biological components from the cancellous bone to the perforated structure and the cage 1430.

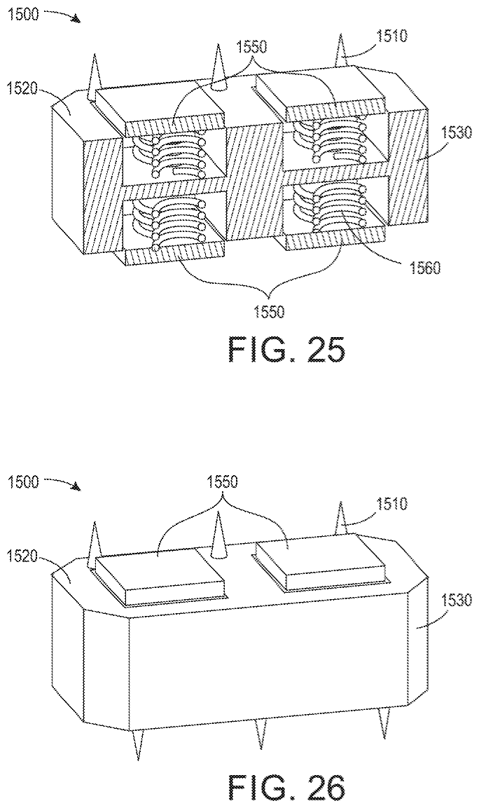

FIG. 25 illustrates a cut-away view of another embodiment of intervertebral fusion mechanism 1500. As illustrated in FIG. 25, the intervertebral fusion mechanism 1500 includes a cage 1530, cancellous bone feeder anchors 1510, and a scaffold structure 1520 on a surface of the cage 1530 that engages the vertebra. The cage 1530 may have openings or voids, as illustrated, which can be filled with bone chips, bone patty, etc. to enhance the fusion.

The intervertebral fusion mechanism 1500 further includes bone growth promotion trays 1550. The bone growth promotion trays 1550 may be filled autograft/allograft/bone/bone substitute or any other bone growth material.

Springs 1560 are located within the cage 1530 to bias the bone growth promotion trays 1550 outwardly so as to compress the bone growth promotion trays 1550 and the bone growth material contained therein against the vertebra.

The strength of the springs 1560 is chosen to apply the appropriate pressure/stress between the bone growth promotion trays 1550 (and the bone growth material contained therein) and the vertebra to promote or enhance bone growth between the vertebra, the intervertebral fusion mechanism 1500, and the surroundings thereof. This promotion of bone growth enables fusion throughout the cage 1530 and the intervertebra space.

In other words, the springs 1560 push the bone growth promotion trays 1550 outwardly to enable the bone growth promotion trays 1550 and the bone growth material contained therein to be compressed against the vertebra to promote bone growth. The remaining portions of the cage 1530 and the cancellous bone feeder anchors 1510 are constructed of scaffold like structures.

As noted above, the cage 1530 forms a fusion device. The cage 1530 may include solid frame members (not shown) to provide strength and support between the two adjacent vertebrae.

It is noted that cancellous bone feeder anchor bases 1510 may be integral with the frame members of the cage 1530.

The cage 1530 may include scaffold portions, located between frame members, to provide a transportation, connection, and/or support mechanism for growing bone thereon. The scaffold portions are constructed to create a plurality of voids (holes or pores) where bone can grow therein so that the scaffold, when the bone is completely formed therearound, is located within the formed bone. In other words, the scaffold portions are porous, and the pores may be interconnected, to promote bone growth in and around the scaffolding portions.

The scaffolding portions of the cage 1530 are formed of titanium or a nickel/titanium or any other material that can serve as a conduit to cells and nutrition.

The cancellous bone feeder anchor 1510 forms perforated devices. The cancellous bone feeder anchor 1510 may include structural reinforcement members (not shown) to provide strength. However, the cancellous bone feeder anchor 1510 forms an effective porous surface area to interact with the cancellous bone, the greater the porous surface area, the more effective that the cancellous bone feeder anchor 1510 can promote bone growth in the intervertebral space between the two adjacent vertebrae. The bone growth in and around the feeder anchor and in the intervertebral space between the two adjacent vertebrae provides strength and support between the disc cage 1530 and the two adjacent vertebrae.

As noted above, the cancellous bone feeder anchor 1510 includes a perforated structure to provide a scaffold or channel for promoting the transference of nutrients, bone cells, vascular cells, and other biological components from the cancellous bone to the cage 1530 and the volume surrounding the cage 1530 so that bone grows in and around the cage 1530.

The perforated structure may be constructed from titanium or a nickel/titanium or tantalum or cobalt chrome alloy or polyetheretherketone or a polyetheretherketone covered alloy, with a plurality of perforations (voids or holes) which promote the transference of nutrients, cells, and other biological components from the cancellous bone to the cage 1530. In other words, the perforated structure is porous, with interconnecting pores, to promote the transference of nutrients, cells, and other biological components from the cancellous bone to the perforated structure and the cage 1530.

FIG. 26 illustrates another view of the intervertebral fusion mechanism 1500 of FIG. 25. As illustrated in FIG. 26, the intervertebral fusion mechanism 1500 includes a cage 1530, cancellous bone feeder anchors 1510, and a scaffold structure 1520 on a surface of the cage 1530 that engages the vertebra. The cage 1530 may have openings or voids, as illustrated, which can be filled with bone chips, bone patty, etc. to enhance the fusion.

The intervertebral fusion mechanism 1500 further includes bone growth promotion trays 1550. The bone growth promotion trays 1550 may be filled autograft/allograft/bone/bone substitute or any other bone growth material.

Springs (not shown) are located within the cage 1530 to bias the bone growth promotion trays 1550 outwardly so as to compress the bone growth promotion trays 1550 and the bone growth material contained therein against the vertebra.

The strength of the springs is chosen to apply the appropriate pressure/stress between the bone growth promotion trays 1550 (and the bone growth material contained therein) and the endplate of the vertebra to promote or enhance bone growth between the vertebra and the intervertebral fusion mechanism 1500. This promotion of bone growth enables fusion throughout the cage 1530 and the intervertebra space.

In other words, the springs push the bone growth promotion trays 1550 outwardly to enable the bone growth promotion trays 1550 and the bone growth material contained therein to be compressed against the vertebra to promote bone growth. The remaining portions of the cage 1530 and the cancellous bone feeder anchors 1510 are constructed of scaffold like structures.

It is noted that in the various embodiments described above, predrilling may be needed to enable proper penetration of the anchors into the cancellous bone.

An intervertebral fusion mechanism includes a disc cage including a scaffolding structure to support bone growth; and a porous cancellous bone feeder anchor, operatively connected to the disc cage, for providing a biological material transference interface between cancellous bone and the disc cage.

The disc cage may form a volume, the scaffolding structure being located within the volume to support bone growth. The disc cage may include an outer surface, the scaffolding structure being located on the outer surface to support bone growth. The disc cage may include an outer surface to interface with an endplate of a vertebra, the scaffolding structure being located on the outer surface to support bone growth between the endplate of the vertebra and the outer surface of the disc cage. The porous cancellous bone feeder anchor may include a perforated structure to support bone growth. The perforated structure may comprise titanium, a nickel/titanium alloy, tantalum, a cobalt chrome alloy, polyetheretherketone, and/or a polyetheretherketone covered alloy. The scaffolding structure may comprise titanium and/or a nickel/titanium alloy.

The scaffolding structure may include a plurality of voids to support bone growth. The perforated structure may be porous, with interconnecting pores, to promote the transference of nutrients, cells, and other biological components from cancellous bone to the perforated structure and the disc cage. The disc cage may include a bone growth promotion tray; the bone growth promotion tray interfacing with an endplate of a vertebra. The disc cage may include an endplate to provide strength at an interface between the disc cage and an endplate of a vertebra. The endplate may be a ring structure. The endplate may include pores to support bone growth. The endplate may include a scaffolding structure to support bone growth. The porous cancellous bone feeder anchor may include a drill structure for penetrating an endplate of a vertebra and cancellous bone.

An intervertebral fusion mechanism includes a disc cage; endplate interfaces for interfacing with endplates of vertebrae; a cage support frame; and a plurality of porous cancellous bone feeder anchors, operatively connected to the disc cage, for providing a biological material transference interface between cancellous bone and the disc cage; each endplate interface including openings such that each opening corresponds to a porous cancellous bone feeder anchor; the plurality of porous cancellous bone feeder anchors being biased to extend from the disc cage; the opening being in a first position, the first position being offset from the corresponding porous cancellous bone feeder anchor to prevent the corresponding porous cancellous bone feeder from extending from the disc cage.

The endplate interfaces may be rotatable so that the opening moves from the first positon to a second position when the endplate interface is rotated, the second position being aligned with the corresponding porous cancellous bone feeder anchor to allow the corresponding porous cancellous bone feeder to extend from the disc cage. The endplate interfaces may be movable so that the opening moves from the first positon to a second position when the endplate interface is moved, the second position being aligned with the corresponding porous cancellous bone feeder anchor to allow the corresponding porous cancellous bone feeder to extend from the disc cage. The cage support frame may be a hollow cylinder structure to provide strength between endplates of vertebrae, the disc cage being located therein. The cage support frame may be porous to promote bone growth.

The intervertebral fusion mechanism may include a spring mechanism to bias the porous cancellous bone feeder anchor to extend from the disc cage. The porous cancellous bone feeder anchors may be configured to have a spring-like characteristic. The disc cage may include scaffolding structure to support bone growth. The cage support frame may be porous to support bone growth. The endplate interfaces may be porous to support bone growth. The plurality of porous cancellous bone feeder anchors may include a perforated structure to support bone growth. The perforated structure may be porous, with interconnecting pores, to promote the transference of nutrients, cells, and other biological components from cancellous bone to the perforated structure and the disc cage. The endplate interfaces may include bone growth promotion trays; the bone growth promotion trays interfacing with an endplate of a vertebra.

An intervertebral fusion mechanism includes a disc cage; endplate interfaces for interfacing with endplates of vertebrae; a cage support frame; a plurality of porous cancellous bone feeder anchors, operatively connected to the disc cage, for providing a biological material transference interface between cancellous bone and the disc cage; and a balloon mechanism, operatively connected to the plurality of porous cancellous bone feeder anchors; each endplate interface including openings such that each opening corresponds to a porous cancellous bone feeder anchor; the plurality of porous cancellous bone feeder anchors extending from the opening when the balloon mechanism is inflated.

The cage support frame may be a hollow cylinder structure to provide strength between endplates of vertebrae, the disc cage being located therein. The cage support frame may be porous to promote bone growth. The disc cage may include scaffolding structure to support bone growth. The cage support frame may be porous to support bone growth. The endplate interfaces may be porous to support bone growth. The plurality of porous cancellous bone feeder anchors may include a perforated structure to support bone growth. The perforated structure may be porous, with interconnecting pores, to promote the transference of nutrients, cells, and other biological components from cancellous bone to the perforated structure and the disc cage. The endplate interfaces may include bone growth promotion trays; the bone growth promotion trays interfacing with an endplate of a vertebra.

An intervertebral fusion mechanism includes a disc cage; endplate interfaces for interfacing with endplates of vertebrae; a cage support frame; and a plurality of porous cancellous bone feeder anchors, operatively connected to the disc cage, for providing a biological material transference interface between cancellous bone and the disc cage; the cage support including tool openings for enabling a tool to interface with a porous cancellous bone feeder anchor; each endplate interface including openings such that each opening corresponds to a porous cancellous bone feeder anchor; the plurality of porous cancellous bone feeder anchors extending from the opening when a tool interfaces therewith.

The cage support frame may be a hollow cylinder structure to provide strength between endplates of vertebrae, the disc cage being located therein. The cage support frame may be porous to promote bone growth. The disc cage may include scaffolding structure to support bone growth. The cage support frame may be porous to support bone growth. The endplate interfaces may be porous to support bone growth. The plurality of porous cancellous bone feeder anchors may include a perforated structure to support bone growth. The perforated structure may be porous, with interconnecting pores, to promote the transference of nutrients, cells, and other biological components from cancellous bone to the perforated structure and the disc cage. The endplate interfaces may include bone growth promotion trays; the bone growth promotion trays interfacing with an endplate of a vertebra.

A method of constructing an intervertebral fusion mechanism having an endplate interface and a plurality of porous cancellous bone feeder anchors includes (a) mapping micro and macro structures of cancellous bone; (b) mapping a topography of an endplate of a vertebra; (c) locating the plurality of porous cancellous bone feeder anchors based upon the mapped micro and macro structures of cancellous bone and cortical bone of a vertebra; and (d) forming a topography of an endplate of the intervertebral fusion mechanism to match the mapped topography of the endplate of the vertebra.

The method may form scaffolding structures in the intervertebral fusion mechanism to support bone growth.

A method of constructing an intervertebral fusion mechanism having an endplate interface and a plurality of porous cancellous bone feeder anchors includes: (a) mapping micro structures of cancellous bone; (b) mapping a topography of an endplate of a vertebra; (c) locating the plurality of porous cancellous bone feeder anchors based upon the mapped micro structures of cancellous bone and cortical bone of a vertebra; and (d) forming a topography of an endplate of the intervertebral fusion mechanism to match the mapped topography of the endplate of the vertebra.

The method may form scaffolding structures in the intervertebral fusion mechanism to support bone growth.

A method of constructing an intervertebral fusion mechanism having an endplate interface and a plurality of porous cancellous bone feeder anchors includes (a) mapping macro structures of cancellous bone; (b) mapping a topography of an endplate of a vertebra; (c) locating the plurality of porous cancellous bone feeder anchors based upon the mapped macro structures of cancellous bone and cortical bone of a vertebra; and (d) forming a topography of an endplate of the intervertebral fusion mechanism to match the mapped topography of the endplate of the vertebra.

The method may form scaffolding structures in the intervertebral fusion mechanism to support bone growth.

A method of constructing an intervertebral fusion mechanism having an endplate interface and a plurality of porous cancellous bone feeder anchors includes (a) mapping structures of cancellous bone; (b) mapping a topography of an endplate of a vertebra; (c) locating the plurality of porous cancellous bone feeder anchors based upon the mapped structures of cancellous bone and cortical bone of a vertebra; and (d) forming a topography of an endplate of the intervertebral fusion mechanism to match the mapped topography of the endplate of the vertebra.

The method may form scaffolding structures in the intervertebral fusion mechanism to support bone growth.

It will be appreciated that variations of the above-disclosed embodiments and other features and functions, or alternatives thereof, may be desirably combined into many other different systems or applications. Also, various presently unforeseen or unanticipated alternatives, modifications, variations, or improvements therein may be subsequently made by those skilled in the art which are also intended to be encompassed by the description above.

1. An intervertebral fusion mechanism comprising:
a disc cage including a scaffolding structure to support bone growth; and
a porous cancellous bone feeder anchor, operatively connected to said disc cage, for providing a biological material transference interface between cancellous bone and said disc cage.

2. The intervertebral fusion mechanism, as claimed in claim 1, wherein said disc cage forms a volume, said scaffolding structure being located within said volume to support bone growth.

3. The intervertebral fusion mechanism, as claimed in claim 1, wherein said disc cage includes an outer surface, said scaffolding structure being located on said outer surface to support bone growth.

4. The intervertebral fusion mechanism, as claimed in claim 1, wherein said disc cage includes an outer surface to interface with an endplate of a vertebra, said scaffolding structure being located on said outer surface to support bone growth between the endplate of the vertebra and said outer surface of said disc cage.

5. The intervertebral fusion mechanism, as claimed in claim 1, wherein said porous cancellous bone feeder anchor includes a perforated structure to support bone growth.

6. The intervertebral fusion mechanism, as claimed in claim 5, wherein said perforated structure comprises titanium.

7. The intervertebral fusion mechanism, as claimed in claim 5, wherein said perforated structure comprises a nickel/titanium alloy.

8. The intervertebral fusion mechanism, as claimed in claim 5, wherein said perforated structure comprises tantalum.

9. The intervertebral fusion mechanism, as claimed in claim 5, wherein said perforated structure comprises a cobalt chrome alloy.

10. The intervertebral fusion mechanism, as claimed in claim 5, wherein said perforated structure comprises polyetheretherketone.

11. The intervertebral fusion mechanism, as claimed in claim 5, wherein said perforated structure comprises a polyetheretherketone covered alloy.

12. The intervertebral fusion mechanism, as claimed in claim 1, wherein said scaffolding structure comprises titanium.

13. The intervertebral fusion mechanism, as claimed in claim 1, wherein said scaffolding structure comprises a nickel/titanium alloy.

14. The intervertebral fusion mechanism, as claimed in claim 1, wherein said scaffolding structure includes a plurality of voids to support bone growth.

15. The intervertebral fusion mechanism, as claimed in claim 5, wherein said perforated structure is porous, with interconnecting pores, to promote the transference of nutrients, cells, and other biological components from cancellous bone to said perforated structure and said disc cage.

16. The intervertebral fusion mechanism, as claimed in claim 1, wherein said disc cage includes a bone growth promotion tray; said bone growth promotion tray interfacing with an endplate of a vertebra.

17. The intervertebral fusion mechanism, as claimed in claim 1, wherein said disc cage includes an endplate to provide strength at an interface between said disc cage and an endplate of a vertebra.

18. The intervertebral fusion mechanism, as claimed in claim 17, wherein said endplate is a ring structure.

19. The intervertebral fusion mechanism, as claimed in claim 17, wherein said endplate includes pores to support bone growth.

20. The intervertebral fusion mechanism, as claimed in claim 17, wherein said endplate includes a scaffolding structure to support bone growth.

21. The intervertebral fusion mechanism, as claimed in claim 1, wherein said porous cancellous bone feeder anchor includes a drill structure for penetrating an endplate of a vertebra and cancellous bone.

22-59. (canceled)
